(19) United States
(12) Patent Application Publication
(10) Pub. No.: US 2022/0395426 A1
(43) Pub. Date: Dec. 15, 2022

(54) SEXUAL AID DEVICE FOR SIMULATING THE FORCE-RESPONSE CHARACTERISTICS OF AN ERECT PENIS

(71) Applicant: Alexander Michael David Winston Myerson, South Burlington, VT (US)

(72) Inventor: Alexander Michael David Winston Myerson, South Burlington, VT (US)

(21) Appl. No.: 17/345,168

(22) Filed: Jun. 11, 2021

(51) Int. Cl. A61H 19/00

(52) U.S. Cl. CPC A61H 19/32; A61H 2201/163; A61H 2201/1652

(57) ABSTRACT

A sexual aid device for simulating the force-response characteristics of an erect penis is disclosed. The sexual aid device provides realistic posterior and anterior force-response characteristics, which stimulate a partner by providing posterior and anterior forces that are experienced inside of and at the egress of an orifice of the partner. Such realistic posterior and anterior force-response characteristics include pressing back and forth in a compressive and/or prying motion against and into many areas of a partner's orifice whereby sexual pleasure-producing nerve endings and/or bundles are stimulated.

### TECHNICAL FIELD

The subject matter of the present invention relates generally to a sexual aid device and more particularly to a sexual aid device for simulating the force-response characteristics of an erect penis for sexual enhancement.

### BACKGROUND

Engaging in enjoyable penetrative sexual intercourse is a primary desire, motivation, and reward in many cultures and relationships. Unfortunately, the means to achieve such experiences are often lacking, causing poor experiences and failed relationships. The realities of gender, desire, physical response, and phallic size and/or shape are common reasons for the failure or meagerness of enjoyment between one or more partners in such intercourse.

Designs for existing phallic devices have typically focused on the attachment of the phallus to the body without major concern for realistic posterior and anterior force-response characteristics. Such devices are limited in the types and amount of pleasure they can provide, as well as the amount of sexual realism they can simulate. The pleasure provided by existing phallic devices derives from orthogonal penetration, which stimulates the partner by forcing the dilation of an orifice (e.g., vagina, anus, etc.) by slipping in and out of the orifice in a transverse motion.

However, orthogonal penetration is only one aspect of natural pleasure-creating sexual motion. Existing phallic devices fail to provide realistic posterior and anterior force-response characteristics, which stimulate the partner by posterior and anterior forces experienced inside of and at the egress of an orifice of the partner, such as pressing back and forth in a compressive and/or prying motion against and into many areas of the orifice. Such characteristics in a phallic apparatus are crucial for most sorts of coital enjoyment and play a large role in sexual satisfaction.

Accordingly, there is a need for an improved sexual aid device for simulating the force-response characteristics of an erect penis for sexual enhancement.

### SUMMARY

To address the foregoing problems, in whole or in part, and/or other problems that may have been observed by persons skilled in the art, the present disclosure provides compositions and methods as described by way of example as set forth below.

In one aspect, a sexual aid device is provided for simulating the force-response characteristics of an erect penis, comprising:

a back portion;

a torso portion; and

a phallus, wherein the phallus comprises a phallus outer layer, a phallus middle layer, and a phallus inner layer;

wherein the back portion is connected to the phallus via a curved back-phallus connector and the torso portion is connected to the phallus via a torso-phallus connector; and further wherein the curved back-phallus connector comprises an opening configured to allow a user who has a scrotum to insert their scrotum through the opening.

In some aspects, the back portion and/or the torso portion are padded and/or comprise natural or synthetic cushioning. In other aspects, the curved back-phallus connector and/or the torso-phallus connector comprise metal. In other aspects, the phallus outer layer and/or the phallus inner layer comprise silicone or another body-safe and realistic material. In other aspects, the phallus middle layer is rigid and comprises a rigid material such as metal. In other aspects, the torso-phallus connector is curved. In other aspects, the back portion and the torso portion are connected to the curved back-phallus connector and the torso-phallus connector, respectively, via an industrial adhesive. In other aspects, the curved back-phallus connector and the torso-phallus connector are each connected to the phallus by an industrial adhesive or a snap-clip mechanism.

In some aspects, when connected, the back portion and the curved back-phallus connector are configured to function as a single unit and provide realistic force-response to posterior-oriented forces upon the phallus, wherein the force-response maintains the phallus firmly in place against posterior-oriented pressure from or upon the coital partner, thereby resulting in enhanced sexual realism and pleasure for the partner.

In some aspects, when connected, the torso portion and the torso-phallus connector are configured to function as a single unit and provide realistic force-response to anterior-oriented forces upon the phallus, wherein the force-response maintains the phallus firmly in place against anterior-oriented pressure from or upon the coital partner, thereby resulting in enhanced sexual realism and pleasure for the partner.

In some aspects, when connected, the phallus, the back portion, the curved back-phallus connector, the torso portion, and the torso-phallus connector are configured to allow the sexual aid device to function as a single unit of firmly affixed parts.

In some aspects, the sexual aid device further comprises means to hold the sexual aid device in place on a user's body, wherein the means comprise one or more straps, belts, suspenders, cords, clips, hooks, wraps, garments, and/or harnesses.

In some aspects, the sexual aid device further comprises a double-sided dildo, a simulated sexual cavity providing tight force-response to an inserted penis, means for lubrication, means for simulating penile ejaculation, and/or means for one or more electronic methods of sexual stimulation comprising vibration, thrusting, rotation, and/or suction.

In some aspects, the sexual aid device further comprises a mechanically, electronically, digitally, and/or sensorially-controlled tubular slider or circularly-oriented series of sliders within the phallus configured to simulate the experience of a user directly thrusting their penis into an orifice of their partner.

In some aspects, the sexual aid device further comprises mechanically, electronically, digitally, and/or sensorially-controlled means to simulate variation between simulated flaccid and erect states.

In some aspects, the sexual aid device further comprises a sex doll, a sex machine, a sex toy, sex furniture, a sexual simulation device, and/or a sex robot.

In another aspect, a method is provided for using any of the sexual aid devices described above, wherein a user:

a) connects the back portion to the phallus;

b) spreads the user's legs and aligns the back portion with the user's lower-middle back so that the back portion abuts the user's back snugly;

c) swings the curved back-phallus connector through the user's legs so that the torso portion, the torso-phallus connector, and the phallus are in front of the user's body; and

d) connects the torso-phallus connector to the phallus, aligning the torso portion with the user's frontal pelvic region and/or lower ventral torso so that the torso portion abuts the user's body snugly. In some aspects, if the user has a scrotum, the user inserts the scrotum through the opening. In some aspects, if the user has a penis, the user inserts the penis into the phallus so that the penis may make contact with the phallus inner layer. In some aspects, if the user does not have an erect penis to hold up the device while in a standing position, the user lies in a supine position and/or employs means to hold up the sexual aid device comprising one or more straps, belts, suspenders, cords, clips, hooks, wraps, garments, and/or harnesses.

In other aspects, the sexual aid device does not comprise the back portion or the torso portion, and the curved back-phallus connector and the torso-phallus connector are configured such that the posterior and/or anterior force-response of the phallus is connected to buttressing forces against the user's body such that the phallus maintains an erect angle in a realistically steadfast manner when pressed upon with posterior and/or anterior pressure.

In other aspects, the sexual aid device enables the user to engage in penetrative coitus with a partner using the phallus of the sexual aid device. The coitus may include styles and/or positions of coitus which ideally require firm posterior and/or anterior force-resistance from the phallic member upon an orifice of their partner.

In other aspects, the sexual aid device may be utilized in the medical field, for example as part of a treatment for a medical condition and/or cosmetic deficiency and to effect a pleasurable posterior and/or anterior force-response on the patient's penetrated partner.

In other aspects, the sexual aid device may be utilized in the field of physical contraception to lower the risk of pregnancy and/or sexually transmitted diseases as a result of any form of penetrative coitus.

In other aspects, the sexual aid device may be utilized in the fields of wearables and fashion for the outward display of a firmly upright and erect phallus, and/or for preparedness for sexual encounters.

In other aspects, the sexual aid device may be utilized in the field of sexual robotics to effect a pleasurable posterior and/or anterior force-response on the penetrated party. In such aspects, a robot takes the place of a human user/wearer of the sexual aid device, and the back portion and the torso portion of the sexual aid device are incorporated within the mechanical design of the robot such that movement of the robot produces realistic posterior and/or anterior force-response characteristics. Such aspects may also use moving elements which cause the position and angle of the phallus to vary over time, thereby providing different and enhanced sensations as compared to those caused by thrusting alone.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 illustrates a perspective view of a sexual aid device for simulating the force-response characteristics of an erect penis, in accordance with an embodiment of the invention;

FIG. 2 illustrates a side view of the sexual aid device shown in FIG. 1;

FIG. 3 illustrates a top view of the sexual aid device shown in FIG. 1; and

FIG. 4 illustrates a cross-section view of the sexual aid device shown in FIG. 1.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. Therefore, it is to be understood that the subject matter of the present invention is not to be limited to the specific embodiments disclosed and that modifications and other embodiments are intended to be included within the scope of the appended claims.

### Sexual Aid Device

In some embodiments, the subject matter of the present invention provides a sexual aid device for simulating the force-response characteristics of an erect penis. The invention provides realistic posterior and anterior force-response characteristics, which stimulate a partner by providing posterior and anterior forces that are experienced inside of and at the egress of an orifice of the partner. Such realistic posterior and anterior force-response characteristics include pressing back and forth in a compressive and/or prying motion against and into many areas of a partner's orifice whereby sexual pleasure-producing nerve endings and/or bundles are stimulated. In some embodiments, a sexual aid device for simulating the force-response characteristics of an erect penis is provided, comprising a synthetic phallus affixed to a user's body. Posterior and anterior force-response stiffness of the phallus are buttressed against the user's back and torso, respectively, such that the position and force-response characteristics of the phallus simulate a strongly erect penis. During penetrative coitus, the anterior and posterior force of the phallus against a partner's coital orifice provides a stiff response for enhanced sexual pleasure in the partner. The penetrated partner achieves a simulated feeling of coital engagement with the user, whereby the sexual aid device enhances sexual pleasure relating to penis size, shape, erection firmness, and posterior and anterior force response strength, thereby enhancing relational satisfaction. The user's sexual pleasure and/or relational satisfaction is also enhanced by enabling the user to provide such enhanced sexual pleasure to the partner.

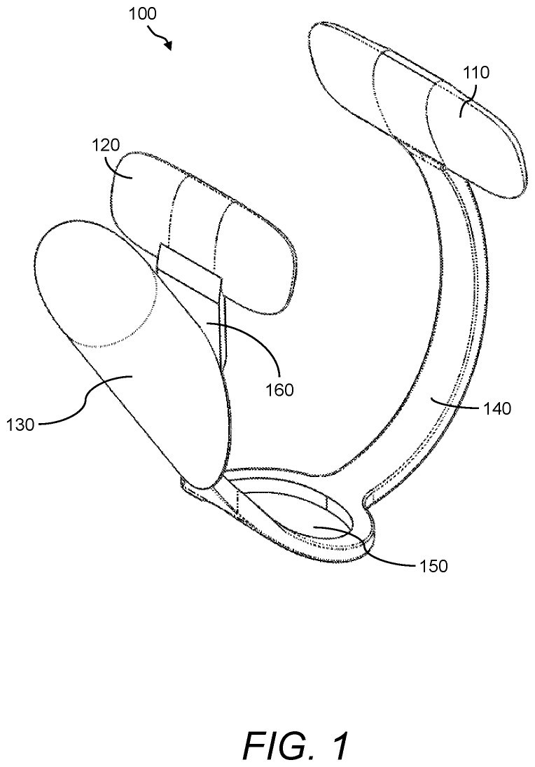

Referring now to FIG. 1, a perspective view is provided of a sexual aid device 100 for simulating the force-response characteristics of an erect penis, in accordance with an embodiment of the invention. Sexual aid device 100 comprises a back portion 110, a torso portion 120, and a phallus 130. The back portion 110 is connected to the phallus 130 via a curved back-phallus connector 140. The curved back-phallus connector 140 comprises an opening 150 configured to allow a user who has a scrotum to insert their scrotum through the opening 150. The torso portion 120 is connected to the phallus 130 via a torso-phallus connector 160.

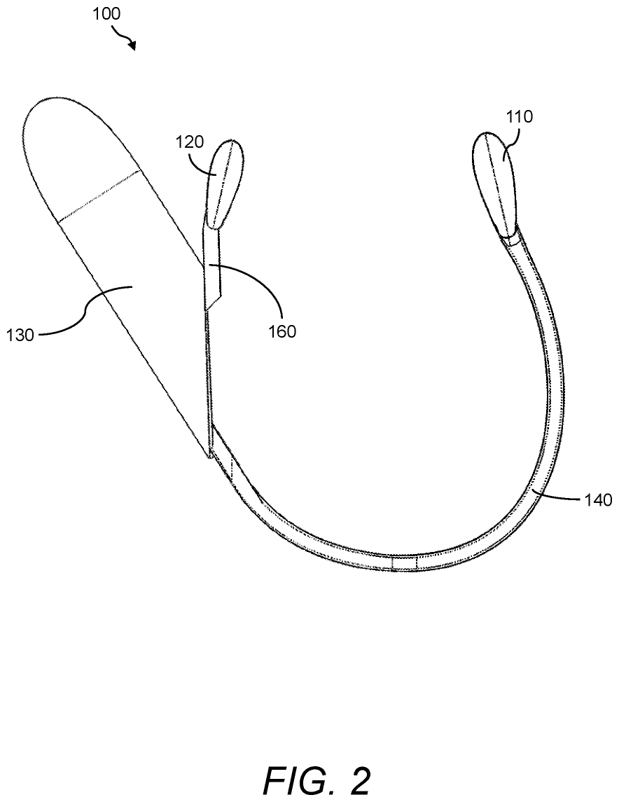

Referring now to FIG. 2, a side view is provided of the sexual aid device shown in FIG. 1. Sexual aid device 100 comprises back portion 110, torso portion 120, and phallus 130. Back portion 110 is connected to phallus 130 via curved back-phallus connector 140. Torso portion 120 is connected to phallus 130 via torso-phallus connector 160.

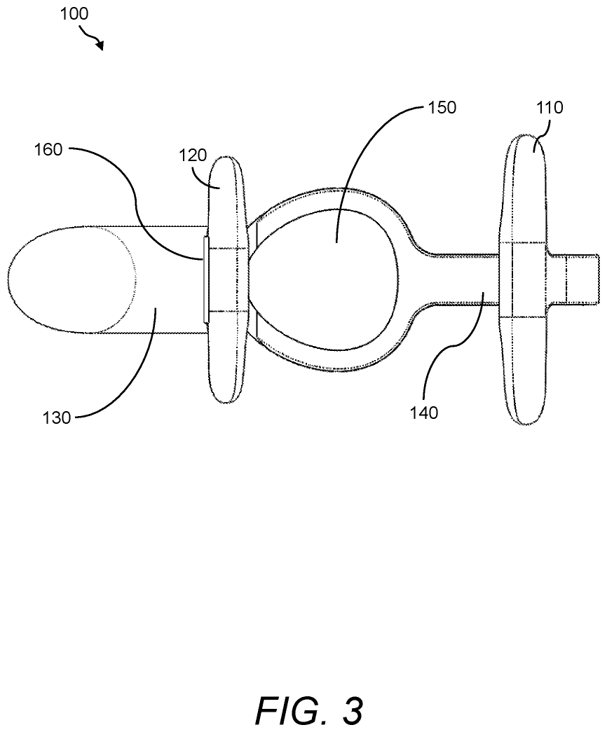

Referring now to FIG. 3, a top view is provided of the sexual aid device shown in FIG. 1. Sexual aid device 100 comprises back portion 110, torso portion 120, and phallus 130. Back portion 110 is connected to phallus 130 via curved back-phallus connector 140. Curved back-phallus connector 140 comprises opening 150 configured to allow a user who has a scrotum to insert their scrotum through opening 150. Torso portion 120 is connected to phallus 130 via torso-phallus connector 160.

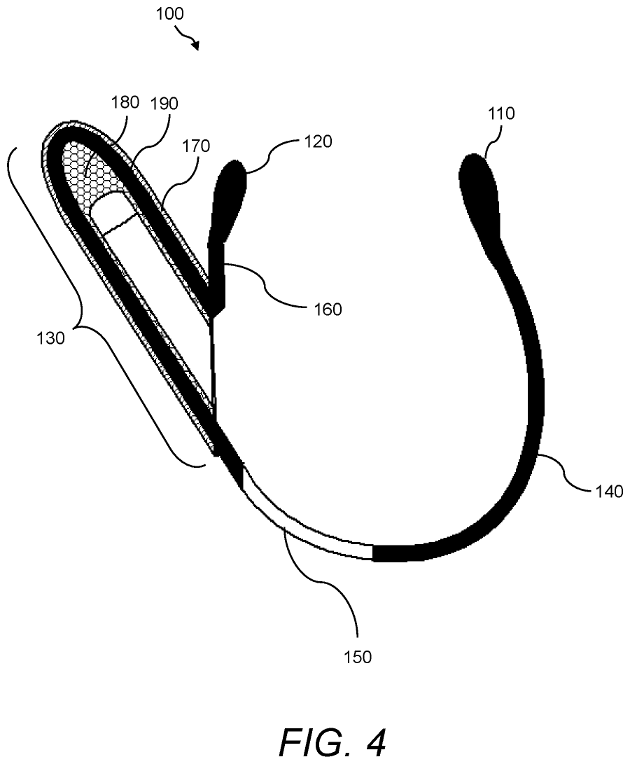

Referring now to FIG. 4, a cross-section view is provided of the sexual aid device shown in FIG. 1. Sexual aid device 100 comprises back portion 110, torso portion 120, and phallus 130. Back portion 110 is connected to phallus 130 via curved back-phallus connector 140. Curved back-phallus connector 140 comprises opening 150 configured to allow a user who has a scrotum to insert their scrotum through opening 150. Torso portion 120 is connected to phallus 130 via torso-phallus connector 160. Phallus 130 comprises a phallus outer layer 170, a phallus inner layer 180, and a phallus middle layer 190.

In one embodiment, back portion 110 and torso portion 120 may each be padded and/or comprise natural or synthetic cushioning. Phallus middle layer 190, curved back-phallus connector 140, and torso-phallus connector 160 may comprise metal. Further, phallus outer layer 170 and phallus inner layer 180 may comprise silicone or another body-safe and realistic material. Phallus middle layer 190 may be rigid and may comprise a rigid material such as metal.

In another embodiment, the torso-phallus connector 160 is curved.

In another embodiment, back portion 110 and torso portion 120 may each be connected to curved back-phallus connector 140 and torso-phallus connector 160, respectively, via any suitable means, including but not limited to an industrial adhesive.

When connected, back portion 110 and curved back-phallus connector 140 function as a single unit, providing realistic force-response to posterior-oriented forces upon phallus 130. This response maintains phallus 130 firmly in place against posterior-oriented pressure from or upon the coital partner, resulting in enhanced sexual realism and pleasure for that partner.

When connected, torso portion 120 and torso-phallus connector 160 function as a single unit, providing realistic force-response to anterior-oriented forces upon phallus 130. This response maintains the phallus firmly in place against anterior-oriented pressure from or upon the coital partner, resulting in enhanced sexual realism and pleasure for that partner.

In another embodiment, curved back-phallus connector 140 and torso-phallus connector 160 may each be connected to phallus 130 via any suitable means, including but not limited to an industrial adhesive, a snap-clip mechanism, or the like. When connected, phallus 130, back portion 110, curved back-phallus connector 140, torso portion 120, and torso-phallus connector 160 allow sexual aid device 100 to function as a single unit of firmly affixed parts. In another embodiment, sexual aid device 100 may be held in place on the user's body via any suitable means, including but not limited to straps, belts, suspenders, cords, clips, hooks, wraps, garments, harnesses, and the like.

In another embodiment, sexual aid device 100 comprises additional components and/or differently-shaped elements. Such additional components and/or differently-shaped elements may increase sexual stimulation for the user and/or their partner and may include, but are not limited to, a double-sided dildo, a simulated sexual cavity providing tight force-response to an inserted penis, lubrication, a mechanism and fluid for simulating penile ejaculation, and various electronic methods of sexual stimulation such as by vibration, thrusting, rotation, and/or suction. The use of a mechanically, electronically, digitally, and/or sensorially-controlled tubular slider or circularly-oriented series of sliders within phallus 130 to simulate the experience of the user directly thrusting their penis into an orifice of their partner would also enhance stimulation. The use of a mechanically, electronically, digitally, and/or sensorially-controlled mechanism to refine, develop, augment, and/or supplement sexual aid device 100 for the purpose of variation between simulated flaccid and erect states would improve or enhance realism, wearability, usability, marketability, mental stimulation, and overall utility.

In other embodiments, the sexual aid device 100 does not comprise back portion 110 or torso portion 120. Further, curved back-phallus connector 140 and torso-phallus connector 160 may be configured and shaped in any number of ways, so long as the posterior and/or anterior force-response of phallus 130 is connected to the buttressing forces against the body of the user such that the phallus 130 maintains an erect angle in a realistically steadfast manner when pressed upon with posterior and/or anterior pressure (i.e., in the manner of a strongly erect penis).

In one embodiment, the user of sexual aid device 100 connects back portion 110 to phallus 130 using a metal snap-clip mechanism. The user then spreads their legs and aligns back portion 110 with their lower-middle back so that back portion 110 abuts their back snugly. The user swings curved back-phallus connector 140 through their legs so that torso portion 120, torso-phallus connector 160, and phallus 130 are in front of their body. If the user has a scrotum, they insert the scrotum through opening 150. If the user has a penis, they insert the erect or flaccid penis into phallus 130, so that their penis may make contact with phallus inner layer 180. The user connects torso-phallus connector 160 to phallus 130 using a metal snap-clip mechanism, aligning torso portion 120 with the user's frontal pelvic region and/or lower ventral torso so that torso portion 120 abuts their body snugly.

The user wearing sexual aid device 100 may lie supine if they do not have an erect penis to hold up the device in a standing position. The user may utilize any number of possible methods for holding sexual aid device 100 in place on the body for use in a non-supine position, including the use of means to hold up the sexual aid device comprising one or more straps, belts, suspenders, cords, clips, hooks, wraps, garments, and/or harnesses.

The user of sexual aid device 100 may then engage in penetrative coitus with a partner using sexual aid device 100 for penetration instead of a natural phallus. The coitus may include styles and/or positions of coitus which ideally require firm posterior and/or anterior force-resistance from the phallic member upon an orifice of their partner.

In other embodiments, sexual aid device 100 may be utilized in applications that do not require a living user. For example, sexual aid device 100 may be used on/with, for example, a sex doll, sex machine, sex toy, sex furniture, sexual simulation device, and/or sex robot to effect a pleasurable posterior and/or anterior force-response on the penetrated party.

In other embodiments, sexual aid device 100 may be utilized in the medical field, for example as part of a treatment for a medical condition and/or cosmetic deficiency and to effect a pleasurable posterior and/or anterior force-response on the patient's penetrated partner.

In other embodiments, sexual aid device 100 may be utilized in the field of physical contraception to lower the risk of pregnancy and/or sexually transmitted diseases as a result of any form of penetrative coitus.

In other embodiments, sexual aid device 100 may be utilized in the fields of wearables and fashion for the outward display of a firmly upright and erect phallus, and/or for preparedness for sexual encounters.

In other embodiments, sexual aid device 100 may be utilized in the field of sexual robotics to effect a pleasurable posterior and/or anterior force-response on the penetrated party.

In such aspects, a robot takes the place of a human user/wearer of the sexual aid device, and the back portion and the torso portion of the sexual aid device are incorporated within the mechanical design of the robot such that movement of the robot produces realistic posterior and/or anterior force-response characteristics. Such embodiments may use moving elements which cause the position and angle of phallus 130 to vary over time and thus provide different and enhanced sensations as compared to those caused by thrusting alone.

In other embodiments, one or more of the elements described herein as utilizing an industrial adhesive or a snap-clip mechanism may alternatively utilize a press fit or snap fit mechanism, including one or more fasteners such as hook and loop fasteners, Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method, or any other suitable removable connection method as one reasonably skilled in the art could envision to serve the same function.

### Definitions

All anatomical parts and/or regions described herein refer to human anatomical parts and/or regions. These anatomical parts and/or regions include, but are not limited to, skin, bone, penises, scrotums, vaginas, testes, perinea, perineal-anal regions, anuses, coccyges, vaginal fourchette, pelvic regions, pubic regions, etc.

As used herein, the term “orifice” (e.g., as in “a partner's orifice”) refers to an opening into which a penis, phallus, dildo, vibrator, or the like can be inserted for penetrative sex such as a vagina, an anus, or a mouth.

As used herein, the term “perineum” refers to: (1) the area of skin between the anus and the scrotum, and/or the area of skin between the anus and the fourchette of the vulva. As used herein, the term “perineal-anal region” includes the perineum (as defined above), the anus, the intergluteal cleft, and the buttocks.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A sexual aid device for simulating the force-response characteristics of an erect penis, comprising:
a back portion;
a torso portion; and
a phallus, wherein the phallus comprises a phallus outer layer, a phallus middle layer, and a phallus inner layer;
wherein the back portion is connected to the phallus via a curved back-phallus connector and the torso portion is connected to the phallus via a torso-phallus connector; and further wherein the curved back-phallus connector comprises an opening configured to allow a user who has a scrotum to insert their scrotum through the opening.

2. The sexual aid device of claim 1, wherein the back portion and/or the torso portion are padded and/or comprise natural or synthetic cushioning.

3. The sexual aid device of claim 1, wherein the curved back-phallus connector and/or the torso-phallus connector comprise metal.

4. The sexual aid device of claim 1, wherein the phallus outer layer and/or the phallus inner layer comprise silicone or another body-safe and realistic material.

5. The sexual aid device of claim 1, wherein the phallus middle layer comprises a rigid material.

6. The sexual aid device of claim 1, wherein the torso-phallus connector is curved.

7. The sexual aid device of claim 1, wherein the back portion and the torso portion are connected to the curved back-phallus connector and the torso-phallus connector, respectively, via an industrial adhesive.

8. The sexual aid device of claim 1, wherein when connected, the back portion and the curved back-phallus connector are configured to function as a single unit and provide realistic force-response to posterior-oriented forces upon the phallus, wherein the force-response maintains the phallus firmly in place against posterior-oriented pressure from or upon the coital partner, thereby resulting in enhanced sexual realism and pleasure for the partner.

9. The sexual aid device of claim 1, wherein when connected, the torso portion and the torso-phallus connector are configured to function as a single unit and provide realistic force-response to anterior-oriented forces upon the phallus, wherein the force-response maintains the phallus firmly in place against anterior-oriented pressure from or upon the coital partner, thereby resulting in enhanced sexual realism and pleasure for the partner.

10. The sexual aid device of claim 1, wherein the curved back-phallus connector and the torso-phallus connector are each connected to the phallus by an industrial adhesive or a snap-clip mechanism.

11. The sexual aid device of claim 1, wherein when connected, the phallus, the back portion, the curved back-phallus connector, the torso portion, and the torso-phallus connector are configured to allow the sexual aid device to function as a single unit of firmly affixed parts.

12. The sexual aid device of claim 1, further comprising means to hold the sexual aid device in place on a user's body, wherein the means comprise one or more straps, belts, suspenders, cords, clips, hooks, wraps, garments, and/or harnesses.

13. The sexual aid device of claim 1, further comprising a double-sided dildo, a simulated sexual cavity providing tight force-response to an inserted penis, means for lubrication, means for simulating penile ejaculation, and/or means for one or more electronic methods of sexual stimulation comprising vibration, thrusting, rotation, and/or suction.

14. The sexual aid device of claim 1, further comprising a mechanically, electronically, digitally, and/or sensorially-controlled tubular slider or circularly-oriented series of sliders within the phallus configured to simulate the experience of a user directly thrusting their penis into an orifice of their partner.

15. The sexual aid device of claim 1, further comprising mechanically, electronically, digitally, and/or sensorially-controlled means to simulate variation between simulated flaccid and erect states.

16. The sexual aid device of claim 1, further comprising a sex doll, a sex machine, a sex toy, sex furniture, a sexual simulation device, and/or a sex robot.

17. A method of using the sexual aid device of claim 1, wherein a user:
a) connects the back portion to the phallus;
b) spreads the user's legs and aligns the back portion with the user's lower-middle back so that the back portion abuts the user's back snugly;
c) swings the curved back-phallus connector through the user's legs so that the torso portion, the torso-phallus connector, and the phallus are in front of the user's body; and
d) connects the torso-phallus connector to the phallus, aligning the torso portion with the user's frontal pelvic region and/or lower ventral torso so that the torso portion abuts the user's body snugly.

18. The method of claim 17, further wherein, if the user has a scrotum, the user inserts the scrotum through the opening.

19. The method of claim 17, further wherein, if the user has a penis, the user inserts the penis into the phallus so that the penis may make contact with the phallus inner layer.

20. The method of claim 19, further wherein, if the user does not have an erect penis to hold up the device while in a standing position, the user lies in a supine position and/or employs means to hold up the device comprising one or more straps, belts, suspenders, cords, clips, hooks, wraps, garments, and/or harnesses.
